(19) United States
(12) Patent Application Publication
(10) Pub. No.: US 2026/0210855 A1
(43) Pub. Date: Jul. 23, 2026

(54) METHOD, APPARATUS, AND LIGHT MICROSCOPE FOR TIME-RESOLVED SINGLE PHOTON MEASUREMENT

(71) Applicant: Abberior Instruments GmbH, Gottingen (DE)

(72) Inventor: Andreas SCHONLE, Gottingen (DE)

(21) Appl. No.: 19/445,783

(22) Filed: Jan. 12, 2026

(30) Foreign Application Priority Data
Jan. 20, 2025 (DE) 10 2025 101 783.2

(51) Int. Cl. A61B 5/00

(52) U.S. Cl. CPC G01N 21/6408; A61B 5/7275; G01N 21/6458

(57) ABSTRACT

A method for time-resolved single photon measurement, wherein light pulses are generated by a light source, photons emitted from a sample as a result of the light pulses are detected by a detector and registered by an evaluation device coupled to the detector, wherein detection times of the photons are determined by the evaluation device, wherein pulse times of a part of the light pulses are determined periodically by the evaluation device by evaluating a signal from the light source or a sensor, wherein the pulse times are determined at a lower rate than a repetition rate of the light pulses, wherein pulse times of further light pulses are estimated based on the determined pulse times, and wherein the pulse times are used as reference times for the detection times of the photons, and an apparatus for single photon measurement, a light microscope comprising the apparatus, and a computer program.

### CROSS-REFERENCE TO RELATED APPLICATIONS

The present application claims the benefit of and priority to DE Patent Application Serial No. DE 10 2025 101 783.2, filed Jan. 20, 2025, the entire contents of which is incorporated herein by reference.

### TECHNICAL FIELD

The present disclosure relates to a method for time-resolved single photon measurement, as well as an apparatus and a light microscope for carrying out the method.

### PRIOR ART

Single photon counting is advantageous for various applications, for example in high-resolution light microscopy and optical spectroscopy.

Many confocal fluorescence light microscopes, for example, use detectors with a counting mode in which they accumulate the emission signal by counting individual photons.

When using detectors that not only count photons but also monitor and record the arrival times of individual photons (e.g., by a method known as time-correlated single photon counting (TCSPC)), the lifetimes of emitters can also be determined. For example, this additional information can be used in lifetime imaging to generate a color-coded image of the sample, with the color code reflecting the lifetime values that depend on the local environment of the emitters in the sample. Furthermore, multiple fluorophores in a microscopic image can be distinguished based on their lifetimes, even if their absorption and/or emission spectra overlap.

Specialized detector modules for time-correlated single photon counting (TCSPC) are known from the prior art. These contain photon detectors and electronic circuits coupled to the photon detector that can be used to count the incoming photons and determine the arrival times of the individual photons at the detector.

An overview of TCSPC and detector technology is provided, for example, in the document W. Becker, The bh TCSPC handbook. 8th edition (2019) available on www.becker-hickl.com.

When measuring emission lifetimes, the result of the counting electronics is usually a histogram of the arrival times of the photons, the shape of which resembles a temporal course of the emission decay with a corresponding time constant that yields the emission lifetime of interest.

An example of a fluorescence lifetime imaging (FLIM) method used in a high-resolution light microscope and based on single-photon counting is described in the publication by Marco Castello et al.: “A robust and versatile platform for image scanning microscopy enabling super-resolution FLIM,” Nature Methods, Brief Communication, doi:10.1038/s41592-018-0291-9, published online on Jan. 14, 2019. In the method described in this publication, excitation light that can excite fluorophores is focused on an area of the sample, which is then imaged onto an array of single-photon avalanche photodiodes (SPADs) arranged in a confocal plane. Based on the signal from the photodiodes, the arrival times of the individual photons at the detector are determined by single-photon counting electronics, and the fluorescence lifetime of the fluorophores is determined from histograms of the arrival times.

In time-correlated single photon counting, synchronization pulses and photon pulses are often recorded with by the evaluation electronics. The synchronization pulses indicate the clock of the pulsed laser source, while the photon pulses originate from a single photon detector and indicate the detection of individual photons. In many single-photon counting systems, a time interval to one or more synchronization pulses is determined for each counted photon. This time interval is often transmitted together with the photon pulse to downstream electronics. Calculating and/or transmitting the time interval can contribute to the dead time of the detection system.

As an alternative to directly transmitting a time interval, the detection of a photon can trigger the transmission of the time of a preceding synchronization pulse or several preceding synchronization pulses to downstream electronics.

On the one hand, determining the time interval between photon detection and the synchronization pulses was historically imperative in early TCSPC systems, as it was not yet technically feasible at that time to determine absolute times with the required precision of less than 100 picoseconds.

On the other hand, the time interval between a photon and the preceding laser pulse, assuming that this laser pulse caused the emission of the photon, is an important parameter from which, for example, fluorescence lifetimes can be determined in FLIM experiments.

To reliably determine such a time interval, the timing of the laser pulses must be determined by the evaluation electronics with the same accuracy as the timing of the photon detection. The evaluation electronics have only a limited number of channels for measuring the time of signals and are therefore limited in their bandwidth.

In many measurements, the number of laser pulses per unit of time is greater than the average number of photons detected in the same time interval, meaning that the count rate (detected photons per time) is often lower than the repetition rate (laser pulses per time). The bandwidth of the detection system is therefore limited by the registration and time measurement of the laser pulses—the detection system is thus saturated at a lower count rate than when only the detected photons are registered.

Another source of error in determining the time intervals between photons and laser pulses is the so-called “jitter,” i.e., the temporal dispersion of the laser pulses in the sample and/or the synchronization pulses in the electronics. The actual jitter of the laser pulses is sometimes not accurately represented by the synchronization pulses, since, for example, no assignment or only an incorrect assignment between individual laser pulses and the respective synchronization pulses is possible. In addition, the extent of the electronic jitter can exceed the pulse-to-pulse jitter. Both effects can contribute to measurement errors in determining the time interval between the photon and the assigned laser pulse.

### OBJECTIVE

This gives rise to the objective of providing a method for time-resolved single-photon measurement and a corresponding apparatus that allows the determination of time intervals between detected photons and respective light pulses with improved bandwidth of the detection system and/or reduced measurement error.

### Solution

This objective is attained by the subject matter of the independent claims. Advantageous embodiments result from the dependent claims and are described below.

### DESCRIPTION

A first aspect of the present disclosure relates to a method for time-resolved single-photon measurement, wherein light pulses are generated by a light source, wherein photons emitted from a sample as a result of the light pulses are detected by a detector and registered by an evaluation device coupled to the detector, wherein detection times of the photons are determined by the evaluation device, wherein pulse times of a part of the light pulses are determined periodically by the evaluation device by evaluating a signal from the light source or a sensor, wherein the pulse times are determined at a lower rate than a repetition rate of the light pulses, wherein pulse times of further light pulses are estimated based on the determined pulse times, and wherein the pulse times are used as reference times for the detection times of the photons.

Since, according to the present disclosure, pulse times are measured only for a part of the light pulses and the pulse times of further light pulses are estimated, the bandwidth of the system that can be used for registering and determining the arrival time of the photons is increased.

Furthermore, the measurement error in determining the time intervals between photon detection and the associated light pulse is reduced at least by the lower influence of the measurement of the pulse times. An even greater reduction in the measurement error results, for example, if the pulse-to-pulse jitter of the light pulses is not exactly reproduced by corresponding electronic synchronization pulses or if the electronic jitter of such synchronization pulses is greater than the pulse-to-pulse jitter of the light pulses.

In particular, the light source may be a pulsed laser. The laser pulses may be generated in a variety of ways, e.g., by combining a CW laser with a pulse picker or acousto-optic modulator. The repetition rate of the light pulses, i.e., the number of light pulses per unit of time, may be in the MHz range for typical applications, but may also be orders of magnitude lower.

The detector may comprise a single light-sensitive element, such as a single-photon avalanche photodiode (SPAD). Alternatively, the detector may comprise several detector elements arranged in a detection plane, which may in particular be individually readable. This is the case, for example, with so-called SPAD arrays.

The evaluation device may be, for example, TCSPC electronics or may comprise such electronics. It may be provided as a unit with the detector (e.g., on a common circuit board and/or enclosed in a common housing) or may be provided separately from the detector and in electrical connection therewith.

The pulse times of the light pulses may be determined, for example, based on electrical synchronization pulses, which are typically generated by the light source or a unit coupled to the light source. Such synchronization pulses may also be generated, for example, based on signals from a sensor in the beam path of the apparatus according to the present disclosure.

The photons emitted as a result of the light pulses may be generated, for example, by fluorophores in the sample being excited to fluorescence by the light pulses. However, the emitted photons may also be reflected or scattered by the sample or objects in the sample, for example. The only decisive factor here is that there is a causal and temporal connection between the light pulses striking the sample and the photons emitted by the sample.

Using the pulse times as reference times for the detection times of the detected photons may mean, in particular, that at least one time interval to a pulse time (in particular, the light pulse immediately preceding the photon) is determined for each of the detected photons. “Immediately preceding” means that no other light pulses occur on the time axis between the photon and the associated light pulse. Optionally, time intervals to several light pulses may also be determined.

According to an embodiment, only the estimated pulse times are used as reference times for the detection times of the photons. The measured pulse times, i.e., those determined by the evaluation device by evaluating a signal from the light source or a sensor, are not used as reference times according to this embodiment. This has the advantage that the error in determining the time intervals between detected photons and associated light pulses does not depend on the error in measuring the pulse times. Rather, this measurement error (in particular any pulse-to-pulse jitter that may be present) can be averaged out, for example, when estimating the pulse times used as reference times from a large number of measured pulse times that do not themselves serve as reference times. The error in determining the time intervals then essentially depends only on the error in measuring the detection times of the photons. As already mentioned above, the method results in a further improvement in measurement accuracy with regard to jitter (pulse-to-pulse jitter of the light pulses in the sample and, if applicable, electronic jitter of the synchronization pulses) if, for example, the light pulses are not perfectly mapped by the synchronization pulses or if the electronic jitter is greater than the pulse-to-pulse jitter of the light pulses in the sample.

According to a further embodiment, at least one preceding pulse time and at least one subsequent pulse time are estimated for each photon detection time. Based on these estimated pulse times, a period and a phase of the respective detection time can be easily determined. The period indicates, in particular, between which successive light pulses the photon occurred, and the phase indicates, in particular, the temporal position of the photon between the light pulses. Estimating only certain pulse times, namely, for example, the pulse times necessary for determining a time interval, has the advantage of lower computational effort compared to estimating all pulse times.

According to an embodiment, the evaluation device comprises data channels and a switching device, wherein output signals from the detector and the light source and/or the sensor are distributed to the data channels by the switching device, wherein detection times of photons and pulse times of the light pulses are determined by the evaluation device using the data channels. In particular, the switching device may be used to alternately connect the detector and the light source or sensor to the same data channel, so that the evaluation device uses the data channel to alternately determine photon detection times and light pulse times. In this way, the bandwidth of the detection system can be optimally utilized.

According to a further embodiment, a time series of estimated pulse times is created by the evaluation device based on the determined pulse times. From the known repetition rate of the light source and the knowledge of the time interval at which the pulse times were measured, a periodic time series can be synthesized, e.g., by determining the least squares of the deviation from the measured pulse times. The pulse times measured at intervals greater than the pulse-to-pulse interval may be used here, for example, as support points for the time series. Due to the measurement error of the pulse times, the measured pulse times then generally no longer correspond exactly to the corresponding pulse times of the synthesized time series. In particular, the time series is periodic and contains pulse times for each light pulse, so the neighboring synthesized pulse times of the time series have an interval that corresponds to the inverse of the repetition rate of the light source. The synthesis of the entire time series has the advantage that, in particular, the period and phase of a photon detection time can be determined particularly quickly, since neighboring pulse times do not have to be estimated for each photon, but the time series is already known.

According to a further embodiment, the time series is continuously adjusted based on further measured pulse times (determined by evaluating a signal from the light source or a sensor). In this case, all existing measured pulse times or only a part of the measured pulse times can be used for the estimation. If only a part of the measured pulse times is used, e.g., the most recent measured pulse times can be used, in particular in the manner of a running window function. This has the advantage that, for example, temporal fluctuations such as a drift in the repetition rate can be better taken into account and the time series thus better reflects the current conditions in the sample.

According to a further embodiment, the pulse times are used as reference times for the detection times of the photons by determining respective periods and phases of the time series for the detection times of the photons.

According to a further embodiment, the rate of determination of the pulse times is 20% of the repetition rate or less, particularly 10% of the repetition rate or less, more particularly 5% of the repetition rate or less. At a value of 10%, for example, only the pulse time of every tenth light pulse is measured. Depending on the photon count rate, this significantly increases the usable bandwidth of the detection system. The use of only every Nth light pulse can be implemented electronically, e.g., by a so-called divider in the signal line between the input of the synchronization pulses and the downstream evaluation electronics.

According to a further embodiment, first light pulses and second light pulses of different colors are generated by the light source or by the light source and a further light source, wherein the first light pulses and the second light pulses are temporally offset from each other, whereby photons emitted from the sample as a result of the first light pulses and photons emitted from the sample as a result of the second light pulses are detected by the detector or by the detector and a further detector, wherein at least pulse times of a part of the first light pulses and/or a part of the second light pulses are determined by the evaluation device by evaluating the signal. This type of alternating sample illumination and detection is also referred to in the prior art as pulse interleaved method.

In this case, the use of the pulse times as reference times may in particular comprise assigning the detected photons to a first light pulse or a second light pulse. For this purpose, the period of the respective photon in the time series of the first light pulses and second light pulses may be determined, in particular if the repetition rates of the light source (or light sources) and the time delay between the first and second light pulses are selected such that, after a first light pulse, it is highly unlikely that a photon caused by the preceding second light pulse will be detected, and vice versa.

According to a further embodiment, pulse times of the second light pulses are estimated based on determined pulse times of the first light pulses, or vice versa. This may be the case, for example, when two different laser sources are used, one of which has a lower pulse-to-pulse jitter of the laser pulses. In this case, if the delay between the first light pulses and the second light pulses is known, it is advantageous to estimate the pulse times of the second light pulses from the measured pulse times of the first light pulses in order to reduce the measurement error, at least if the temporal change in the delay between the first light pulses and the second light pulses is sufficiently small.

According to a further embodiment, the sample contains emitters, in particular fluorescence emitters, wherein the emitters are excited by the light pulses to emit photons, wherein a lifetime of the emitters is determined by the evaluation device based on the detected photons and the reference times.

The lifetime is defined here as a parameter that indicates the typical (or average) time after which a photon is emitted following a light pulse. In the case of fluorescence emission by the emitters in response to excitation light, the emission lifetime is the fluorescence lifetime. If the light emission can be described as a monoexponential process, for example, the emission lifetime is defined in particular as the inverse of the time constant of a monoexponential decay. For an ensemble of emitters, this lifetime describes the time after which the light intensity of the light emission has decreased to a fraction of 1/e of the initial intensity value. In the case of a single emitter, the emission of a photon after being triggered by the light pulse follows a probability distribution that can be described, for example, by a monoexponential decay, and the emission lifetime is the time after which the probability that a particular emitter has emitted a photon is equal to 1-1/e.

The term “emitter” used here refers to a molecule, molecular complex, or particle that emits electromagnetic radiation, particularly in the visible, infrared, or ultraviolet range, when excited by external light. The emitter may, for example, be a fluorophore or be coupled (i.e., covalently or non-covalently bound) to a fluorophore that emits fluorescent light when excited by a light pulse of suitable wavelengths. Alternatively, the emitter may be, for example, a quantum dot. In addition to fluorescence, other mechanisms are also conceivable within the scope of the present disclosure for exciting the at least one emitter to emit a photon. For example, emitted photons may be a result of light scattering. Another example is photoactivation, which may consist of converting the at least one emitter from a first, e.g., non-fluorescent state or dark state to a second, light-emitting state, e.g., by the at least one emitter emitting fluorescence in response to excitation light in the second state. In this case, the excitation light that excites the at least one emitter in the second state may originate from the light pulses that also activate the at least one emitter, or the excitation light may originate from another source, in particular wherein the excitation light may have a different wavelength than the activation light.

A second aspect of the present disclosure relates to an apparatus for time-resolved single-photon measurement, in particular according to a method according to the first aspect, wherein the apparatus comprises a detector (e.g., a single-photon avalanche photodiode, SPAD, or an SPAD array) that is configured to detect photons emitted by a sample as a result of light pulses, and an evaluation device (e.g., TCSPC electronics) coupled to the detector, the evaluation device being configured to determine detection times of the photons, wherein the evaluation device is configured to periodically determine pulse times of a part of the light pulses at a rate lower than a repetition rate of the light pulses by evaluating a signal from a light source (e.g., a laser) or a sensor, to estimate pulse times of further light pulses based on the determined pulse times, and to use the pulse times as reference times for the detection times of the photons.

A third aspect of the present disclosure relates to a light microscope comprising a light source (e.g., a laser) configured to generate light pulses, and an apparatus for time-resolved single-photon measurement according to the second aspect.

A fourth aspect of the present disclosure relates to a non-transitory computer readable medium for storing computer instructions for time-resolved single photon measurement that when executed by at least one processor causes the at least one processor to perform the method according to the first aspect.

Further features and advantages of the apparatus according to the second aspect, the light microscope according to the third aspect, and the computer program according to the fourth aspect result analogously from the explanations relating to the method according to the first aspect.

### BRIEF DESCRIPTION OF THE FIGURES

FIG. 1 shows an exemplary time series of light pulses and photons to illustrate an embodiment of the present disclosure;

FIG. 2 shows an apparatus for single photon measurement according to an embodiment of the present disclosure;

FIG. 3 shows a light microscope according to an embodiment of the present disclosure;

FIG. 4 shows a light microscope according to a further embodiment of the present disclosure.

### DESCRIPTION OF THE FIGURES

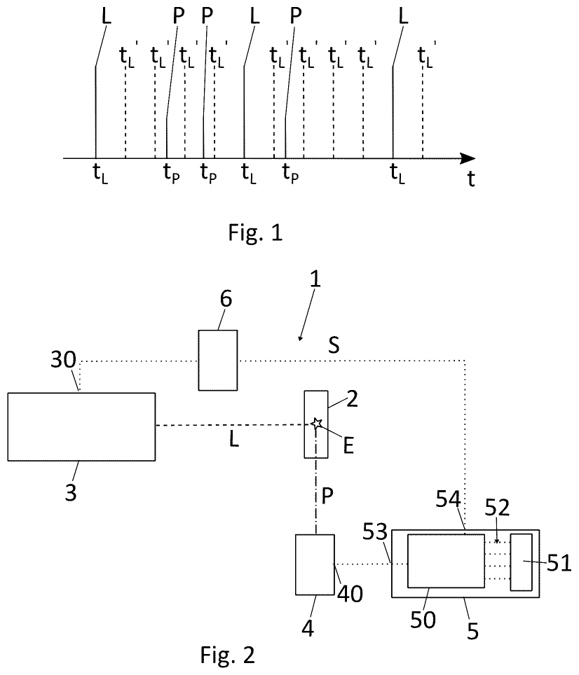

FIG. 1 schematically shows an exemplary time series of light pulses L generated by a light source, e.g., a laser, illuminating a sample, and photons P detected by a detector, which are emitted by the sample, e.g., by emitters in the sample as a result of the light pulses L. The photons P may be, for example, fluorescence photons emitted by fluorophores in the sample, wherein the light pulses excite the fluorophores to fluoresce. The detection times t_{P} for the photons P are plotted on the time axis.

FIG. 1 shows only those light pulses L whose pulse times t_{L} were determined by evaluating a signal from the light source 3 or a sensor. According to the present disclosure, further pulse times t_{L}′ are estimated based on the pulse times t_{L} recorded by measurement (dashed lines in FIG. 1). According to the example shown in FIG. 1, only the pulse timing t_{L} of every fifth light pulse L is determined by measurement; the remaining pulse timings t_{L}′ are estimated, e.g., by fitting a time function (using the known repetition rate of the light source) to the measured pulse timings t_{L}.

In particular, the entire time series of light pulses L is reconstructed from the pulse times t_{L} recorded by measurement through estimation, i.e., even for those light pulses L whose pulse times t_{L} were measured, pulse times t_{L}′ are estimated.

The estimated pulse times t_{L}′ are used as reference times for the detection times t_{P} of the photons. This means, in particular, that time intervals between the detection times t_{P} and the pulse times t_{L}′ are determined.

FIG. 2 shows an example of an embodiment of an apparatus 1 for single photon measurement according to the present disclosure. The apparatus 1 comprises a light source 3, e.g., a laser, which generates light pulses L. The light pulses L strike a sample 2 and excite emitters E in the sample 2, in particular to fluorescence, so that these emit photons P. The apparatus 1 also comprises a detector 4 that is configured to detect the photons P. A signal output 40 of the detector 4 is coupled to a first signal input 53 of an evaluation device 5 of the apparatus 1, so that the evaluation device 5 can receive signals or data from the detector 4. The evaluation device 5 comprises a computing unit 51 that is configured to determine detection times t_{P} for individual photons P detected by the detector 4. The signal runs from the first signal input 53 via a switching device 50 and via one of several data channels 52 to the computing unit 51.

The light source 3 may also be provided as a separate component, i.e., it does not have to be part of the apparatus 1.

The light source 3 transmits synchronization pulses S via a signal output 30, which is connected via a divider 6 to a second signal input 54 of the evaluation device 5, which synchronization pulses indicate pulse times t_{L} of the light pulses L generated by the light source 3. The divider 6 is configured to transmit only a part of the synchronization pulses S, e.g., every fifth or every tenth synchronization pulse S, to the second signal input 54 of the evaluation device 5.

The switching unit 50 of the evaluation device 5 distributes signals of the detector 4 arriving via the first signal input 53 and synchronization pulses S of the light source 30 arriving via the second signal input 54 to data channels 52, via which they reach the computing unit 51.

The computing unit 51 is configured to determine pulse times t_{L} based on the synchronization pulses S and to estimate further pulse times t_{L}′ based on the pulse times t_{L}. Furthermore, the computing unit 51 is configured to use the pulse times t_{L}′ as reference times for the detection times t_{P} of the photons, i.e., in particular, to determine time intervals between the detection times t_{P} and associated pulse times t_{L}′, e.g. in order to determine a period and a phase of a photon detection with respect to the time series of the light pulses L. Based on this information, e.g. emission lifetimes of a fluorophore may then be determined by the computing unit 51 or a separate computing unit.

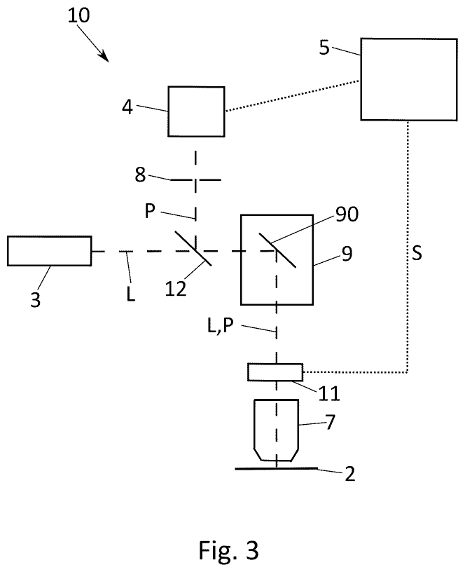

FIG. 3 schematically shows an example of a light microscope 10 according to the present disclosure. In this (non-limiting) embodiment, the light microscope 10 is configured as a confocal laser scanning microscope and comprises a light source 3, in particular a laser, for generating light pulses L. The light of the light pulses L passes through a beam splitter 12, a scanning device 9 with a movable scanning mirror 90, and an objective 7 into a sample 2, wherein the light is focused into the sample 2, in particular by the objective 7, and the scanning device 9 is configured to scan the focus of the light over or through the sample 2 by moving (in particular rotating) the scanning mirror 90. Emitters may be arranged in the sample 2, which are excited by the light pulses L and then emit photons P. The emitted photons P are collected by the objective 7, de-scanned by the scanning device 9 and reflected by the beam splitter 12, in particular a dichroic mirror, into a detection beam path. The detection beam path contains a pinhole 8 and a detector 4 for detecting the photons P. The detector 4 is connected to an evaluation device 5. The detector 4 and the evaluation device 5 form an apparatus 1 for single-photon measurement according to the present disclosure, which may be configured, for example, as shown in FIG. 2 and described above. In contrast to the arrangement shown in FIG. 2, however, it is not the light source 3 but a sensor 11 arranged in the beam path between the scanning device 9 and the objective 7 that generates synchronization pulses S, which are transmitted to the evaluation device 5. The synchronization pulses S are evaluated by the evaluation device 5 to determine pulse times t_{L} of the light pulses L. Based on the pulse times t_{L}, further pulse times t_{L}′ are then estimated by the evaluation device 5. The sensor 11 may, of course, also be arranged at another suitable location in the beam path of the light microscope 10. Furthermore, the light microscope 10 according to the present disclosure may, of course, also contain an apparatus 1 according to FIG. 2, i.e., the synchronization pulses S may also originate from the light source 3.

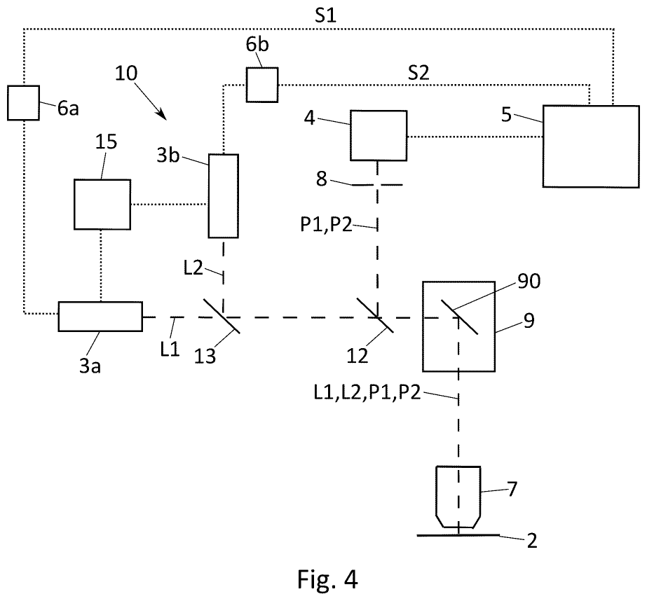

FIG. 4 shows a further embodiment of the light microscope 10 according to the present disclosure. As in the embodiment according to FIG. 3, it is also a confocal laser scanning microscope. Identical components are designated with identical reference numerals in FIG. 3 and FIG. 4. In this regard, reference is made to the description of FIG. 3.

The light microscope 10 shown in FIG. 4 comprises a first light source 3a for generating first light pulses L1 and a second light source 3b for generating second light pulses L2, wherein the first light pulses L1 and the second light pulses L2 have different wavelength components. The first light pulses L1 and the second light pulses L2 can be used, for example, to excite first and second emitters of different types in the sample 2, so that the first emitters emit first photons P1 and the second emitters emit second photons P2 (in particular of different wavelengths). The first light pulses L1 and the second light pulses L2 are coupled together into the main beam path of the light microscope 10 at a beam combiner 13. The first light pulses L1 and the second light pulses L2 are emitted from the respective light sources 3a, 3b with a time delay (so-called pulse-interleaved excitation, PIE), wherein a time delay between the first light pulses L1 and the second light pulses L2 can be set using a control unit 15 connected to the first light source 3a and the second light source 3b.

The first photons P1 and the second photons P2 are collected by the objective 7, de-scanned by the scanning device 9, and reflected by the beam splitter 12 into the detection beam path of the light microscope 10. In the detection beam path, the first photons P1 and the second photons P2 pass through a pinhole 8 to a detector 4, which detects both the first photons P1 and the second photons P2. Alternatively, the first photons P1 and the second photons P2 may also be split in the detection beam path based on their wavelengths using an additional beam splitter and detected by separate detectors (not shown).

A signal output of the detector 4 is connected to the evaluation device 5. The evaluation device 5 determines respective detection times t_{P} for the photons P1, P2. The first light source 3a generates first synchronization pulses S1 and the second light source 3b generates second synchronization pulses S2, which are transmitted via an optional first divider 6a and an optional second divider 6b, respectively, to respective inputs of the evaluation device 5. Alternatively, the first and second synchronization pulses S1, S2 may also be transmitted to the same input of the evaluation device 5, wherein corresponding identifiers are also transmitted which encode information about the origin of the synchronization pulses S1, S2 from the first light source 3a or the second light source 3b, respectively. The evaluation device 5 then determines the pulse times t_{L} of the first light pulse L1 and the second light pulse L2 based on the first and second synchronization pulses S1, S2. Subsequently, further pulse times t_{L}′ may be estimated based on the measured pulse times t_{L}. Based on the detection times t_{P} and the pulse times t_{L} of the first light pulse L1 and the second light pulse L2, it is then possible to determine in particular which of the light pulses L1, L2 triggered the corresponding photon P1, P2, i.e., which emitter species it originated from. The repetition rates of the light sources 3a, 3b and the pulse delay are selected in particular so that a first photon P1 caused by the first light pulse L1 is highly likely to occur before the subsequent second light pulse L2, so that the photons can be clearly assigned to a first light pulse L1 or a second light pulse L2 based on the period of their detection in the time series of light pulses L1,L2, i.e., as first photons P1 or second photons P2.

As an alternative to the configuration shown in FIG. 4, the evaluation device 5 may be connected only to the first light source 3a or the second light source 3b and receive only first synchronization pulses S1 or only second synchronization pulses S2. Based on the first synchronization pulses S1, the evaluation device 5 may estimate pulse times t_{L}′ for the first light pulses L1 and the second light pulses L2. This is particularly useful if there is no significant fluctuation in the delay between the first light pulses L1 and the second light pulses L2.

### LIST OF REFERENCE SIGNS

1 Apparatus for single photon measurement
2 Sample
3 Light source
3a First light source
3b Second light source
4 Detector
5 Evaluation device
6 Divider
6a First divider
6b Second divider
7 Objective
8 Pinhole
9 Scanning device
10 Light microscope
11 Sensor
12 Beam splitter
13 Beam combiner
15 Control unit
30 Signal output
40 Signal output
50 Switching device
51 Arithmetic unit
52 Data channel
53 First signal input
54 Second signal input
90 Scan mirror
L Light pulse
L1 First light pulse
L2 Second light pulse
P Photon
P1 First photon
P2 Second photon
S Synchronization pulse
S1 First synchronization pulse
S2 Second synchronization pulse
t_{L} Determined pulse time
t_{L}′ Estimated pulse time
t_{P} Detection time

1. A method for time-resolved single-photon measurement, wherein light pulses are generated by a light source, wherein photons emitted from a sample as a result of the light pulses are detected by a detector and registered by an evaluation device coupled to the detector, wherein detection times of the photons are determined by the evaluation device,
wherein the pulse times of a part of the light pulses are determined periodically by the evaluation device by evaluating a signal from the light source or a sensor, wherein the pulse times are determined at a lower rate than a repetition rate of the light pulses, wherein the pulse times of further light pulses are estimated based on the determined pulse times, and wherein the pulse times are used as reference times for the detection times of the photons.

2. The method according to claim 1, wherein using the pulse times as the reference times for the detection times of the photons comprises determining for each of the detected photons a time interval to a light pulse immediately preceding the detected photon.

3. The method according to claim 1, wherein only the estimated pulse times are used as the reference times for the detection times of the photons.

4. The method according to claim 3, wherein for each of the detection times of the photons, at least one preceding pulse time and at least one subsequent pulse time are estimated.

5. The method according to claim 1, wherein the evaluation device comprises data channels and a switching device, wherein output signals from the detector and the light source and/or the sensor are distributed to the data channels by the switching device, wherein the detection times of the photons and the pulse times of the light pulses are determined by the evaluation device using the data channels.

6. The method according to claim 1, wherein a time series of the estimated pulse times is created by the evaluation device based on the determined pulse times.

7. The method according to claim 6, wherein the time series is continuously adjusted based on further measured pulse times determined by evaluating the signal from the light source or the sensor.

8. The method according to claim 6, wherein the pulse times are used as the reference times for the detection times of the photons by determining respective periods and phases of the time series for the detection times of the photons.

9. The method according to claim 1, wherein the rate of determination of the pulse times is 20% of the repetition rate or less.

10. The method according to claim 1, wherein the rate of determination of the pulse times is 10% of the repetition rate or less.

11. The method according to claim 1, wherein the rate of determination is 5% of the repetition rate or less.

12. The method according to claim 1, wherein first light pulses and second light pulses of different colors are generated by the light source or by the light source and a further light source, wherein the first light pulses and the second light pulses are temporally offset from each other, wherein the photons emitted from the sample as a result of the first light pulses and the photons emitted from the sample as a result of the second light pulses are detected by the detector or by the detector and the further detector, wherein at least the pulse times of a part of the first light pulses and/or a part of the second light pulses are determined by the evaluation device by evaluating the signal.

13. The method according to claim 12, wherein the pulse times of the second light pulses are estimated based on the determined pulse times of the first light pulses or the pulse times of the first light pulses are estimated based on the determined pulse times of the second light pulses.

14. The method according to claim 12, wherein using the pulse times as the reference times comprises assigning the detected photons to one of the first light pulses or the second light pulses.

15. The method according to claim 1, wherein the sample contains emitters, wherein the emitters are excited by the light pulses to emit the photons, wherein a lifetime of the emitters is determined by the evaluation device based on the detected photons and the reference times.

16. The method according to claim 15, wherein the emitters are fluorescence emitters.

17. An apparatus for time-resolved single photon measurement, comprising
a detector configured to detect photons emitted by a sample as a result of light pulses,
an evaluation device coupled to the detector, the evaluation device being configured to determine detection times of the photons,
wherein the evaluation device is configured to periodically determine pulse times of a part of the light pulses at a lower rate than a repetition rate of the light pulses by evaluating a signal from a light source or a sensor, to estimate pulse times of further light pulses based on the determined pulse times, and to use the pulse times as reference times for the detection times of the photons.

18. A light microscope comprising a light source configured to generate light pulses and an apparatus for time-resolved single photon measurement according to claim 17.

19. A non-transitory computer readable medium for storing computer instructions for time-resolved single photon measurement that when executed by at least one processor causes the at least one processor to perform the method according to claim 1.
